(19) United States
(12) Patent Application Publication
(10) Pub. No.: US 2020/0141842 A1
(43) Pub. Date: May 7, 2020

(54) Antibody Pen

(71) Applicant: Brigham Young University, Provo, UT (US)

(72) Inventors: Richard Watt, Provo, UT (US); Jamon Armitstead, Provo, UT (US); Annie Armitstead, Provo, UT (US); Lara Grether, Maple Valley, WA (US); Kelly Pitts, Thornton, CO (US)

(21) Appl. No.: 16/677,356

(22) Filed: Nov. 7, 2019

Related U.S. Application Data
(60) Provisional application No. 62/757,029, filed on Nov. 7, 2018.

(51) Int. Cl. G01N 1/31; G01N 33/577; G01N 33/543; C07K 16/18

(52) U.S. Cl. CPC G01N 1/31; G01N 33/577; B43K 7/00; C07K 16/18; G01N 33/54306; G01N 33/54386

(57) ABSTRACT

Conventional methods for applying the antibodies to the test paper include using expensive striping machines. The present disclosure addresses a need for easier means for preparing test paper for LFI assays. A pen is provided that may include a housing. The housing may include a distal end and a proximal end. The pen may include a tip connected to the distal end of the housing; an ink compartment disposed between the distal end and the proximal end of the housing; and a solution in the ink compartment. The solution may include an antibody or protein.

### CROSS REFERENCE TO RELATED APPLICATION

This application claims the benefit of the filing date under 35 U.S.C. § 119(e) of U.S. Provisional Patent Application No. 62/757,029 filed on Nov. 7, 2018, which is incorporated by reference herein in its entirety.

### FIELD OF THE TECHNOLOGY

The present disclosure generally relates to deposition of antibodies or biomarkers on surfaces, such as paper, for immunological biomarker/chemical detection and diagnostic applications.

### BACKGROUND

Lateral Flow Immunoassays (LFI) are widely used in disease detection because of the ability to specifically identify biomarkers and other chemical markers. LFI tests require only a small sample of blood, serum, urine, saliva, or other sample containing a biomarker to produce a positive or negative result for the analyte of interest. The detection of the biomarker occurs when the biomarker binds to antibodies that are striped onto the test paper. Current methods for applying the antibodies to the test paper use expensive striping machines which are X-Y plotter type equipment with pumps to deposit an even layer of antibody. Once the sample is added to the test paper, the LFI acts as a “dip stick” that is dipped into a tube with buffer. This buffer carries the sample up through the conjugate pad via capillary action until it passes over the test and control lines prepared with antibodies that specifically bind the biomarker or the antibody. The tag on the conjugate antibody allows visualization of the marker.

### SUMMARY OF THE INVENTION

A pen is provided that may include a housing comprising a distal end and a proximal end; a tip connected to the distal end of the housing; an ink compartment disposed between the distal end and the proximal end of the housing; and a solution in the ink compartment. The solution may include an antibody or protein.

A kit is also provided. The kit may include any pen described herein and a lateral flow immunoassay substrate.

A method of preparing a lateral flow immunoassay substrate is provided. The method may include applying a solution from a pen to a substrate. The pen may include a housing comprising a distal end and a proximal end; a tip connected to the distal end of the housing; an ink compartment disposed between the distal end and the proximal end of the housing. The solution is contained in the ink compartment before applying to the substrate. The solution may include an antibody.

### BRIEF DESCRIPTION OF THE DRAWINGS

FIG. 1 shows the different parts of a typical LFI test strip. The deposition of the test and control lines with the antibody pen is the focus of this invention.

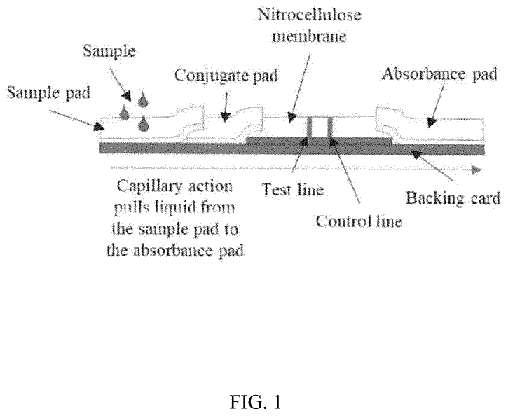

FIG. 2 shows a simplified form of the LFI of FIG. 1.

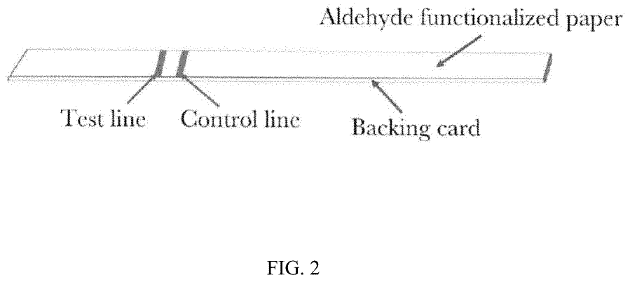

FIG. 3 shows perspective views of a roller ball pen with filter inserted (A); a roller ball pen with filter removed (B); and an embodiment of the pen with a solution (antibody ink) (C). The pen is assembled with the wick inserted.

FIG. 4 shows images of developed western blots using the pen. A) aldehyde functionalized paper. B) Nitrocellulose paper.

FIG. 5 displays the test strips after they had run for 10 minutes.

### DETAILED DESCRIPTION

### Definitions

As used herein the specification, “a” or “an” may mean one or more. As used herein in the claim(s), when used in conjunction with the word “comprising”, the words “a” or “an” may mean one or more than one. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or composition that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features.

Throughout this application, the term “about” is used to indicate that a value includes the standard deviation of error for the device or method being employed to determine the value.

Conventional methods for applying the antibodies to the test paper include using expensive striping machines. The present disclosure addresses a need for easier means for preparing test paper for LFI assays.

The pen described herein was developed as an inexpensive alternative method to accurately and evenly distribute antibody test lines to the LFI tests without requiring the expensive striping machines.

Labs and clinics in developing countries need quick and simple diagnostic tests due to the lack of infrastructure and the high cost of diagnostic equipment. LFI test strips meet many of these diagnostic needs. The present disclosure provides a solution to expensive striping machines by providing a pen that can be used to prepare low-cost LFI test strips. The availability of the “antibody ink” or “biomarker ink” will give the clinicians versatility in their diagnostic testing. Note that antibodies can be lyophilized and remain stable without refrigeration and can be prepared as “antibody ink” by adding water, buffer or other solutions that will work in the pen.

A pen is provided that may include a housing comprising a distal end and a proximal end; a tip connected to the distal end of the housing; an ink compartment disposed between the distal end and the proximal end of the housing; and a solution in the ink compartment. The solution may include an antibody or protein.

In some aspects, the pen may include a wick in contact with the solution in the ink compartment capable of delivering the solution to the tip. The pen may have a longitudinal axis along which the wick is aligned such that a solution in the middle portion of the pen or the proximal end can travel through the wick to the tip at the distal end of the pen. For example, a rollerball pen has a wick or filter that may absorbs the solution containing the antibody or protein and prevents leakage of non-viscous solutions.

In some aspects, the wick may include silk. Silk may stabilize the proteins and antibodies. For relatively unstable proteins, a silk wick or silk filter can be used to stabilize antibodies or proteins.

The tip of the pen may be felt or ball-point. In some aspects, the tip may be spheroidal. Although felt tipped pens and ball point pens may not be the ideal delivery devices, each pen might have advantages in another delivery applications. For instance, dot blot tests with antibodies are performed by spotting a protein or antibody on nitrocellulose paper, and a felt tip pen might be ideal for such an application. Some proteins and antibodies require glycerol or detergents for stabilization, so a ball point pen might be ideal for housing such antibodies or biomarkers inks.

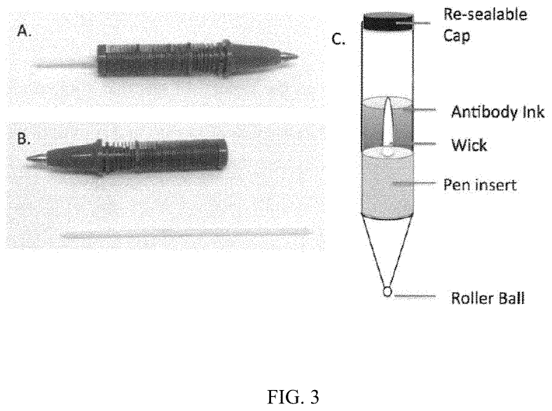

In some aspects, the pen may be a rollerball pen. The rollerball pen is a combination of a fountain pen and a ball point pen. Roller ball pens have a wick or filter that absorbs the antibody ink and prevents leakage of non-viscous solutions (FIGS. 3A & 3B). The wick circumvents the need for adding additional components to the antibody ink buffer that was required in the ball point pen application to prevent ink leakage from the pen. The wick also provides a very even flow of antibody onto the paper. The antibody ink was added to the top of the wick to ensure saturation of the wick and even distribution of the antibody ink on the paper (FIG. 3C). The ball point tip of the roller ball pen further facilitates an even distribution of the antibody ink onto the paper. Therefore, the most even distribution of the antibodies onto the LFI paper have been achieved with the roller ball pen version of the antibody pen.

A plastic, metal, ceramic, or glass housing can be used for the internal components. These might be varied depending on potential interactions with biomolecules and might include additional materials to prevent interactions. For instance, DNA will sometimes bind to glass so if DNA or RNA were used, a metal or plastic or ceramic ball and components might be required.

A lid for the tip of the pen is advantageous, to seal off air and prevent evaporation of the solution. This will facilitate storage of the pen if all of the antibody is not used immediately.

A removable, resealable backing on the proximal end of the pen may facilitate addition of the solution into the pen's ink compartment.

The solution may include an antibody or a biomarker. In some aspects, the solution further include a buffer. Examples of buffers include, but are not limited to, phosphate buffered saline or Tris-HCl or Tris Base. Preferably, the buffer pH may be between 7 and 8. One of ordinary skill in the art would be able to select a buffer that would allow the antibody or biomarker to remain stable in the pen.

Proteins or antibodies can be prepared in lyophilized forms and stored without refrigeration. The solution can be produced by hydrating the protein in water, buffer, or other biologically relevant solutions to stabilize the protein or antibody.

In some aspects, the solution may include a dye. The dye may be conjugated to an antibody or biomarker in the solution. Examples of dyes include, but are not limited to, AlexaFluor dyes, Cyanine dyes, fluorescein dyes, or rhodamine dyes. In some aspects, the dye may be an indicator such as a gold nanoparticle or magnetic particle that can be detected using surface plasmon resonance or magnetic fields.

In some aspects, the dye may be a fading dye to verify the line is properly applied; an invisible ink type dye that can be tested and fades; or fluorescent molecules that are only activated by altered lighting conditions, for example a Europium marker might be used and visualized using a UV lamp to verify even distribution of the solution on the test and control lines.

The solution may include an antibody stabilizing agent. Examples of stabilizing agents include, but are not limited to, glycerol, sucrose, bovine serum albumin, sodium azide, or gelatin.

In some aspects, the solution may include rheology modifying agents such as glycerol. For example, some pen designs may require a certain solution viscosity to prevent leakage of the solution from the tip or uneven flow of the solution from the tip of the pen.

A kit is also provided. The kit may include any pen described herein and a lateral flow immunoassay substrate. The kit may include different colored tubes to assist the user in not confusing the test and control line antibody pens. Also, a variety of convenient methods to label the pens in ways that the solution content are not confused.

The lateral flow immunoassay substrate may be an aldehyde-functionalized printer paper. In some aspects, the aldehyde-functionalized printer paper may be made by soaking a piece of printing paper in 0.03 M KIO4 solution for about 2 hours at about 65 degrees Celsius. The paper is then removed from heat and rinsed by dipping it in fresh deionized water. The paper is then blotted dry and allowed to further dry at about 35 degrees Celsius for at least 12 hours.

The paper becomes more hydrophilic after aldehyde functionalization, allowing tests to run in about 10 minutes or less.

A method of preparing a lateral flow immunoassay substrate is provided. The method may include applying a solution from a pen to a substrate. The pen may include a housing comprising a distal end and a proximal end; a tip connected to the distal end of the housing; an ink compartment disposed between the distal end and the proximal end of the housing. The solution is contained in the ink compartment before applying to the substrate. The solution may include an antibody.

The substrate may be an aldehyde-functionalized printer paper or nitrocellulose paper.

In some aspects, the lateral flow immunoassay substrate may be prepared by measuring and marking two lines about 5.0 mm apart near the middle of the aldehyde functionalized paper. One line may be the test line that contains a capture antibody that recognizes the biomarker and the other line is the control line that binds the Fc portion of the conjugated antibody. In conventional LFIs, the absorbance pad helps with the capillary action. In the present application, the test lines are positioned near the middle of the aldehyde functionalized paper, so the extra paper length can act as an absorbance pad and facilitate capillary action.

Using a straight edge or stencil and an antibody pen the antibody solution is striped across the paper, following the markings made previously. Stripe the control protein solution 5.0 mm above the test protein solution.

In some aspects, a stencil may guide the deposition of the antibodies onto the proper location of the test and control lines on the LFI paper. In some aspects, the stencil may contain ideal spacing gaps between the test and control lines. The use of a stencil with the pen provides an additional aspect of control to optimize the successful deposition of the test and control lines to the substrate.

The method may include allowing the lines to dry. In some aspects, the method may include placing the striped paper in a shallow dish, filling the dish with blocking solution, and mixing for 1 hour. The blocking solution can be powdered milk, bovine serum albumin, or any abundant protein available in the low resource setting that will not interfere with the specificity of the assay. Perhaps beans or other plant proteins present in the country that is abundant and will not interfere with the human sample assay.

Once the blocking has completed, the paper is dried on a rack at room temperature for about 2 hours, using a clean flat surface to press the paper flat and prevent curling.

The antibody pen described herein can be used in other potential diagnostic platforms as a method to accurately and evenly distribute antibodies or other chemical or biochemical species on a surface.

In summary, the pen acts as a writing instrument to be used to deposit different antibodies/proteins and other chemical solutions onto a surface. Contact and sustained contact with movement rolls the small metal ball in a ballpoint pen, or simple contact in a felt-tipped pen, which draws the protein, chemical, or compound solution from the ink reservoir to the ball or the felt tip, in the ballpoint pen, roller ball pen, or felt-tipped pen respectively. A dot or line of protein, chemical, or compound solution ink is deposited on a surface. This disclosure could be employed in any case where a line of protein, chemical, or compound solution would be useful.

### EXAMPLES

### Example 1

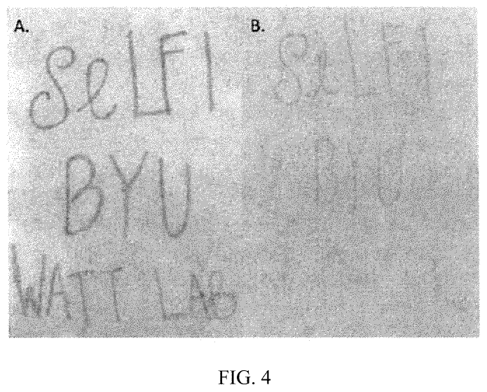

The pen was tested using the method of western blotting. The pen was used to deposit goat-anti hCG onto A) aldehyde functionalized paper or B) nitrocellulose paper. After the antibody ink dried, the paper was treated with hCG in 5% dry milk TBS-T buffer at 25° C. while shaking for 1 hour. After washing, mouse-anti-hCG was added. The mouse-anti-hCG was conjugated to gold nanoparticles to visualize the ink, and the sample was incubated 1 hour followed by washing in distilled water. The initial deposition of the antibody ink was visualized (FIG. 4). The pen was more effective in depositing the antibody ink on the aldehyde functionalized paper than on the nitrocellulose paper.

### Example 2

In this experiment, the pen was used to dispense Goat anti-Mouse (0.5 mg/ml in 1XPBS) in a horizontal line on nitrocellulose membrane (GE/Whatman, FF120HP). The membrane was then tested as a half strip (test strip with no conjugate pad) in 50 uL of buffer with 5 uL of anti-Mouse gold conjugate.

### 1. Filling the Pen:

a. Obtain a clean wick that is saturated with diH2O.
b. Remove the colored pen tip from the clear, plastic barrel. Do not damage the metal ballpoint of the pen.
c. Insert the wick into the small hole in the colored pen tip.
d. Pipette ≥100 uL of antibody solution into the clear, plastic barrel of the pen.
e. Replace the colored pen tip with wick into the barrel.

### 2. Dispensing the Pen:

a. Turn the pen tip downward and tap gently to make sure the antibody solution contacts the wick.
b. On a test material, start drawing with the pen until the reagent starts to come out of the tip. At this point, draw at least 2×300 mm lines on a paper towel before dispensing on your nitrocellulose.
c. Align a ruler to where you would like the antibody to be drawn for the test lines.
d. Use the edge of the ruler to direct the pen across the paper. To avoid damaging the nitrocellulose, use as little pressure as possible as you slowly drag the pen along the edge of the ruler. Draw slowly, ensuring that the solution in the pen is flowing, and that it is distributing evenly.
e. Allow the test line to dry before testing.

### 3. Testing

a. Laminate the nitrocellulose 16 mm from the bottom edge of a 60 mm wide backing card. Adhere a 21 mm absorbent pad along the top edge of the card and a 2 mm overlap onto the nitrocellulose. Cut into 5 mm test strips.
b. Pipette 50 uL of running buffer into a glass test tube (1XPBS with 0.1% Tween-20).
c. Pipette 5 uL of an OD 10 gold conjugate into the test tube and gently swirl to mix the sample.
d. Cut off the extra 16 mm of backing card at the bottom of the test strip.
e. Drop the strip into the test tube with the sample and allow the strip to run for 5-10 minutes.

After filling the pen, it took a couple minutes of drawing before the liquid flowed through the tip of the pen. After the liquid was visible, about 8-10 lines were drawn on a scrap piece of nitrocellulose prior to dispensing on the final material.

A ruler was used as a guide for the dispensing pattern.

The result depended on the speed at which the pen moved. The slower the rate, the wider the line. Regardless of how gently the pen was used, it consistently left a visible impression on the nitrocellulose.

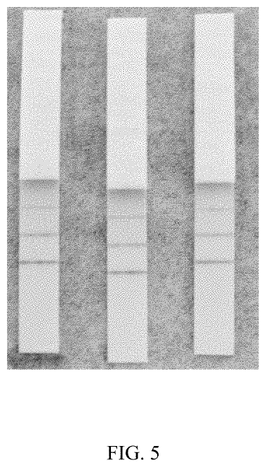

As expected, 3 control lines were visible on the test strips (FIG. 5). The lines appeared lighter towards the absorbent pad due to the flow characteristics of the conjugate and background discoloration. This effect was not due to the dispensing.

One skilled in the art will understand that the embodiment of the present invention as shown in the drawings and described above is exemplary only and not intended to be limiting. Embodiments have been shown and described for the purposes of illustrating the functional and structural principles of the present invention and is subject to change without departure from such principles. Therefore, this invention includes all modifications encompassed within the spirit and scope of the following statements and claims.

What is claimed is:

1. A pen, comprising:
a housing comprising a distal end and a proximal end;
a tip connected to the distal end of the housing;
an ink compartment disposed between the distal end and the proximal end of the housing; and
a solution in the ink compartment, the solution comprising an antibody or protein.

2. The pen of claim 1, further comprising a wick in contact with the solution in the ink compartment capable of delivering the solution to the tip.

3. The pen of claim 2, wherein the wick comprises silk.

4. The pen of claim 1, wherein the tip is spheroidal.

5. The pen of claim 1, wherein the pen is a rollerball pen.

6. The pen of claim 1, wherein the tip comprises felt.

7. The pen of claim 1, wherein the solution further comprises a buffer.

8. The pen of claim 1, wherein the solution further comprises a dye.

9. The pen of claim 1, wherein the solution further comprises an antibody stabilizing agent.

10. A kit, comprising the pen of claim 1 and a lateral flow immunoassay substrate.

11. The kit of claim 10, wherein the pen further comprises a wick in contact with the solution in the ink compartment capable of delivering the solution to the tip.

12. The kit of claim 11, wherein the wick comprises silk.

13. The kit of claim 10, wherein the tip is spheroidal.

14. The kit of claim 10, wherein the pen is a rollerball pen.

15. The kit of claim 10, wherein the tip comprises felt.

16. The kit of claim 10, wherein the solution in the pen further comprises a buffer.

17. The kit of claim 10, wherein the solution in the pen further comprises a dye.

18. The pen of claim 10, wherein the solution in the pen further comprises an antibody stabilizing agent.

19. A method of preparing a lateral flow immunoassay substrate, comprising:
applying a solution from a pen to a substrate;
wherein the pen comprises:
a housing comprising a distal end and a proximal end;
a tip connected to the distal end of the housing;
an ink compartment disposed between the distal end and the proximal end of the housing; and
the solution in the ink compartment, the solution comprising an antibody.

20. The method of claim 19, wherein the substrate is an aldehyde-functionalized printer paper.
